(19) United States
(12) Patent Application Publication
(10) Pub. No.: US 2026/0034382 A1
(43) Pub. Date: Feb. 5, 2026

(54) RADIATION TREATMENT PLAN OPTIMIZATION METHOD AND APPARATUS

(71) Applicant: Siemens Healthineers International AG, Steinhausen (CH)

(72) Inventors: Esa Kuusela, Espoo (FI); Marko Rusanen, Espoo (FI)

(21) Appl. No.: 18/791,665

(22) Filed: Aug. 1, 2024

(51) Int. Cl. A61N 5/10

(52) U.S. Cl. CPC A61N 5/1031; A61N 5/1037; A61N 5/1039; A61N 5/1048; A61N 5/1081; A61N 2005/1089

(57) ABSTRACT

A control circuit accesses a plurality of images of a particular patient (such as, for example, a plurality of computed tomography images), which images are temporally dispersed and collectively depict a particular dynamically-moving part of that particular patient over time. The control circuit then optimizes a first part of a radiation treatment plan as a function of a first one of the plurality of images and optimizes a second, different part of the radiation treatment plan as a function of a second, different one of the plurality of images. The control circuit can then output an optimized radiation treatment plan.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Some portions of the human body are subject to cyclic movement (for example, due to breathing). Such movement can increase the challenges of accurately planning a radiation treatment plan and administering that plan as the position of the target can move during treatment.

### BRIEF DESCRIPTION OF DRAWINGS

The above needs are at least partially met through provision of the radiation treatment plan optimization method and apparatus described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a schematic representation as configured in accordance with various embodiments of these teachings; and

FIG. 4 comprises a schematic representation as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments can serve to facilitate optimizing a radiation treatment plan for a particular patient. By one approach, a control circuit accesses a plurality of images of that particular patient (such as, for example, a plurality of computed tomography images), which images are temporally dispersed and collectively depict a particular dynamically-moving part of the particular patient over time. The control circuit then optimizes a first part of the radiation treatment plan as a function of a first one of the plurality of images and optimizes a second, different part of the radiation treatment plan as a function of a second, different one of the plurality of images. The control circuit can then output an optimized radiation treatment plan.

By one approach, the aforementioned particular dynamically-moving part of the particular patient can comprise at least a part of the particular patient's chest that moves as the particular patient breathes.

In support of the foregoing, and as one optional approach, these teachings will accommodate processing the plurality of images to determine at least one dynamic feature of the particular dynamically-moving part of the particular patient over time, to thereby select which of the plurality of images are to be used when optimizing different parts of the radiation treatment plan.

It will be understood that these teachings are not limited to only using a first and second one of the plurality of images. Instead, as appropriate, these teachings will accommodate separately optimizing each of N different parts of the radiation treatment plan, each as a function of a separate, different one of the plurality of images, where “N” is an integer greater than “2.” That said, these teachings will also accommodate, as appropriate, using a single one of the plurality of images for two or more of the different parts of the radiation treatment plan when appropriate.

By way of example, the aforementioned first part of the radiation treatment plan can correspond to a first control point, the second part of the radiation treatment plan can correspond to a second, different control point, and each of the N different parts of the radiation treatment plan can themselves each correspond to different control points.

By one approach, these teachings can include then administering the optimized radiation treatment plan to the particular patient. The foregoing can comprise synchronizing administration of the optimized radiation treatment plan with observed during-treatment movement of the particular dynamically-moving part of the particular patient (such as, for example, breathing-based movement of the particular patient's chest). By one approach, the foregoing synchronization of administration of the optimized radiation treatment plan can comprise selectively controlling gantry speed while administering the optimized radiation treatment plan.

So configured, these teachings can comprise a dynamic treatment that is undertaken essentially over an entire multi-phase breath cycle. The optimization process can start by deciding how the arc-field is to be delivered over time, i.e., the duration of time each gantry interval will take. Once this timing is done, the optimization can determine at which times the breath-cycle will move into a next phase (i.e., when one should move to use a next three-dimensional computed tomography scan to describe the patient geometry). Once this timing is done, the optimizer dose-engine can be configured so that whenever dose for a control point is calculated, the appropriate corresponding three-dimensional computed tomography image is used. During administration of the treatment plan, the delivery can be synchronized to use the same timing (while still allowing for timing to be adjusted during treatment (for example, by slowing down or speeding up the gantry) to accommodate changes to the patient's movement behavior).

These teachings can provide for better treatment efficiency and/or accuracy. Compared to gating as is sometimes applied in prior art practice, the present teachings allow formulating and applying the treatment plan in a more precise and accommodating way with respect to the breathing cycle. Compared to standard free-breathing planning (using a single leads image), the proposed planning is based on a more realistic estimation of the delivered dose. Allowing the patient to breathe in an unrestricted and more natural manner is also more likely to be comfortable for the patient.

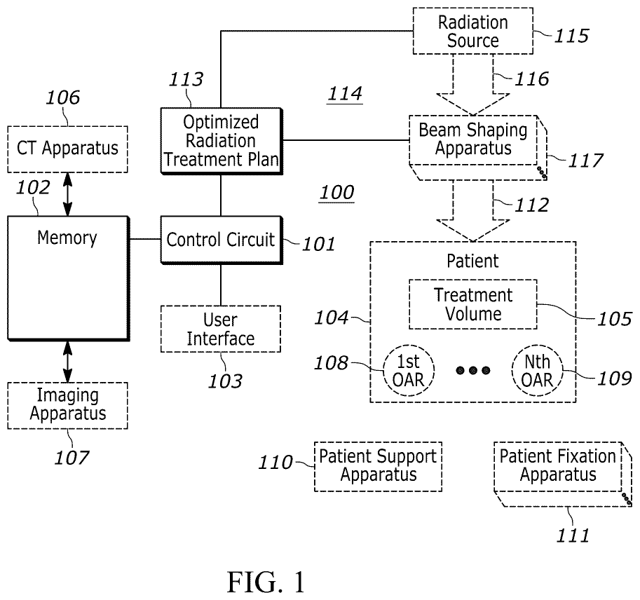

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as three-dimensional computed tomography images, optimization information for a particular patient, and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses and it will be understood that the energy-based treatment platform itself comprises a physical structure.

In a typical application setting the energy-based treatment platform 114 will include an energy source such as a radiation source 115 of ionizing radiation 116. By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

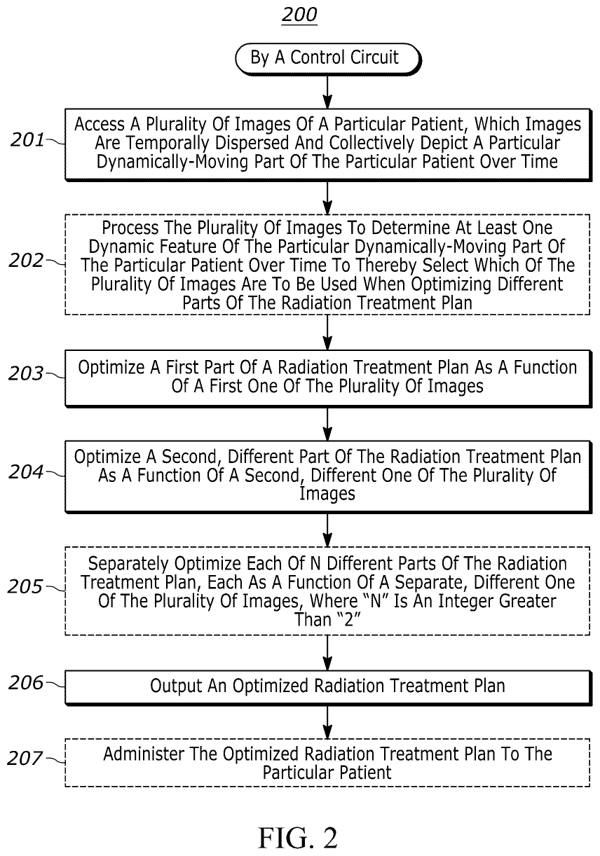

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

At block 201, this process 200 has the control circuit 101 accessing (for example, by accessing the aforementioned memory 102) a plurality of images of the particular patient, which images are temporally dispersed and collectively depict a particular dynamically-moving part of the particular patient over time as a corresponding sequence of images of that movement.

By one approach, but without suggesting any limitations in these regards, that plurality of images may include some computed tomography images or may even be comprised wholly of computed tomography images that are captured, for example, by the aforementioned CT apparatus 106. Those skilled in the art will understand that the expression “computed tomography” refers to a computerized x-ray imaging procedure in which a beam of x-rays is aimed at a patient and rotated around the patient's body to yield signals that are processed by the machine's computer to generate cross-sectional images, sometime referred to as slices. These slices are called tomographic images and can give a clinician more detailed information than conventional x-rays. Once a number of successive slices are collected by the machine's computer, the slices can be digitally stacked to form a three-dimensional (3D) image of the patient that allows for easier identification of basic structures as well as possible tumors or abnormalities.

As noted above, these images are captured over time. By one approach, these images are captured at regular intervals (such as, for example, every 0.25 seconds, 0.50 seconds, 0.75 seconds, 1.00 seconds, or the like). By another approach, if desired, these images may be captured, at least in part, in a more irregular manner (albeit in some time-stamped manner so that the relative timing between images remains known). The latter may comprise, for example, triggering an image capture as a function of real-time monitoring of the particular patient's dynamically moving body part.

These images, separated in time, may be taken over a time interval and may include a complete cycle of the movement of the dynamically moving body part. By another approach, multiple cycles may be captured in this manner and the results utilized to develop, for example, a representative complete movement cycle by selecting images that are most representative of movement during various parts of the movement cycle. By another approach, images from different cycle samples may be processed (for example, by averaging and/or morphing) to develop a composite representative view of particular parts of the movement cycle.

These teachings will accommodate use with a variety of different parts of a patient's body that are subject to dynamic movement (and especially regular movement such as a part (or all) of a patient's chest that moves as the patient breathes.)

At optional block 202, the control circuit 101 can process the plurality of images to determine at least one dynamic feature of the particular dynamically-moving part of the particular patient over time to thereby select which of the plurality of images are to be used when optimizing different parts of the radiation treatment plan. Examples of useful dynamic features can include, but are not limited to, physical movement that at least meets a particular distance threshold. By one approach, one or more such features could be based on relative positions between a target structure and/or one or more organs-at-risk. As one such example, projections of targets and organs-at-risk (calculated, for example, vis a viz the geometry of a given beam direction and a given image) could be assessed to determine how such projections relate to one another. Such a comparison could indicate, for example, a degree of overlap between a target projection and particular organ-at-risk projections. As another example, when no such overlap exists, such a comparison could instead indicate a shortest distance between a target and an organ-at-risk projection.

Beginning at block 203, the control circuit 101 begins the optimization process. Specifically at block 203, the control circuit 101 optimizes a first part of the radiation treatment plan (such as a part of the plan that corresponds to a particular part of a treatment arc) as a function of a first one of the plurality of images. To be clear, this part of the optimization process is not making use of other images from the plurality of images. When, for example, a patient's complete breathing cycle is now represented by six temporally-separated computed tomography images, a part of the radiation treatment plan that corresponds to the beginning of the patient's breathing cycle may only make use of a first computed tomography image that corresponds to that beginning portion of the patient's breathing cycle.

In a somewhat similar manner, at block 204, the control circuit 101 then optimizes a second, different part of the radiation treatment plan as a function of a second, different one of the plurality of images. To continue with the simple example presented immediately above, this step may comprise optimizing a next part of the radiation treatment plan that addresses a portion of the patient's breathing cycle that next follows the beginning portion described above using a next computed tomography image in the temporal sequence of images that represent the patient's breathing cycle.

As presented in optional block 205, the foregoing process can be repeated for additional portions of the radiation treatment plan using temporally subsequent images. In particular, the control circuit 101 can be further configured to separately optimize each of N different parts of the radiation treatment plan, each as a function of a separate, different one of the plurality of images, where “N” is an integer greater than “2.”

By one approach, the aforementioned different parts of the radiation treatment plan can correspond to different control points. For example, the first part of the radiation treatment plan can correspond to a first control point, the second part of the radiation treatment plan can correspond to a second, different control point (such as a next control point that is adjacent to the first control point), and each of the N different parts of the radiation treatment plan can each correspond to different control points. (Control points refer to specific positions or angles at which a radiation beam is turned on or off during treatment. Control points typically serve to help shape the radiation beam and to optimize the dose distribution to target the tumor while sparing surrounding healthy tissues.)

Upon completing the optimization process, at block 206 the control circuit 101 outputs an optimized radiation treatment plan (such as the optimized energy treatment plan 113 described above).

At optional block 207, the control circuit 101 can then administer the optimized radiation treatment plan to the particular patient (using, for example, the aforementioned radiation treatment platform 114).

By one approach, initiation of the treatment occurs when the patient is undergoing the appropriate corresponding phase of the breathing cycle. For example, if the optimized radiation treatment plan begins at the bottom of the patient's breathing cycle, the treatment itself should begin when the patient's real-time breathing cycle is similarly situated. That synchronization can be automated as desired.

It is possible that the patient's anticipated (and planned-for) body movement will not exactly match the previously observed (and planned-for) movement. To accommodate such a situation, these teachings will accommodate configuring the control circuit 101 to administer the optimized radiation treatment plan to the particular patient by synchronizing administration of the optimized radiation treatment plan with observed during-treatment movement of the particular dynamically-moving part of the particular patient. Such synchronization may be achieved, at least in part, by selectively controlling gantry speed while administering the optimized radiation treatment plan. For example, when the patient is breathing faster than was previously observed, the gantry speed may be speeded up to match the plan to the patient's now-observed movement. Similarly, when the patient is breathing slower than was previously observed, the gantry speed may be slowed down to match the plan to the patient's now-observed movement.

By one approach, while monitoring the patient, one can calculate the difference of the phase between the planned timing and the current observed real-time timing. The delivery speed of the plan can then be adjusted accordingly. With this in mind, it can be useful to prepare the plan to not use the fastest available speed of delivery so that it is possible to speed up delivery in order to catch up when the patient is breathing faster than expected.

By one approach, the aforementioned planning can be configured to occur in two phases, a first timing phase and a follow-on optimization phase.

During the timing phase, by one approach it may be assumed that the gantry is moving at a constant speed that is somewhat below the maximum speed, and that any other axis of movement/change will be restricted during optimization so as not to affect the treatment time. By this approach, the timing of treatment delivery can be determined during a preprocessing step prior the actual optimization. Using this approach, the timing need not be changed during optimization.

By another approach, the timing phase can be repeated after the initial optimization to also determine intervals where the amount of monitor units requires the delivery to be slowed down. Then subsequently, the optimization phase can be repeated using the modified timing information.

These teachings will also accommodate combining timing with the optimization phase. In such a case, if desired, the timing can be recalculated (in whole or in part) during each optimization iteration.

By one approach, the optimization phase can make use of typical volumetric modulated arc therapy optimization loops except that the total dose evaluation and the cost function evaluation can follow the approaches described herein, while additionally selecting a proper patient image when evaluating a particular dose.

Further details that comport with these teachings will now be presented. It will be understood that the specific details of these examples are intended to serve an illustrative purpose and are not intended to suggest any particular limitations with respect to these teachings.

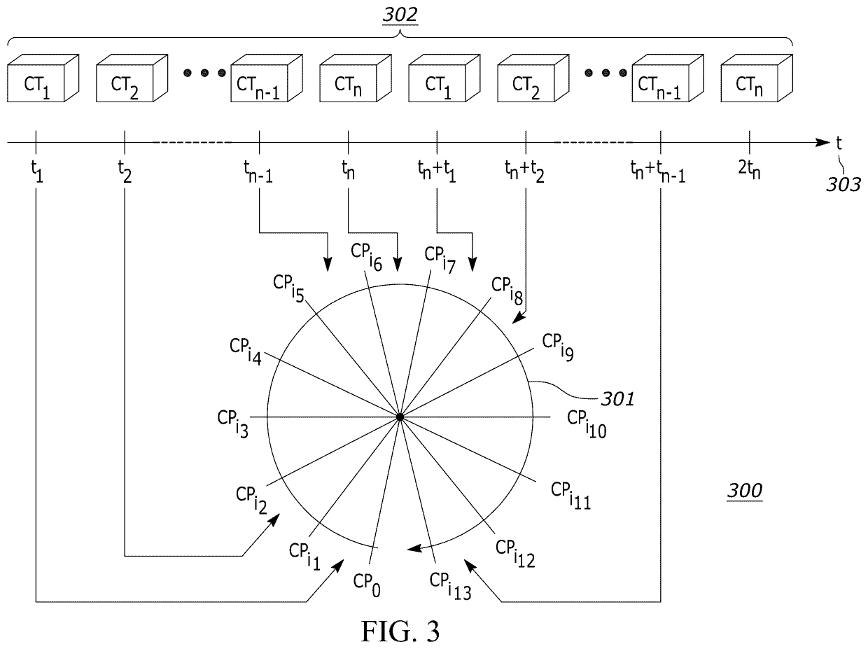

FIG. 3 presents a schematic view 300 of how an arc field 301 is scheduled to be treated during a breath cycle. The whole breath cycle is divided into n phases, each having its own computed tomography image 302 associated with it. There are also approximative times (t) 303 associated with the middle point of each phase. In this example, the breath cycles are assumed to repeat following the same pattern, so the 1^{st} phase is repeated after the n^{th} phase (although the time-instances continue increasing). Similarly, the delivery of the arc-fields is split into intervals described by pivotal control-points CPᵢ_{ⱼ} (where index iⱼ refers to the control point index that was treated at the moment when the j^{th} phase change is occurring). In a preparatory phase, the iⱼ's are selected so that they match with corresponding tⱼ's (assuming a reasonable average gantry rotation speed).

When dose is calculated for sector CPᵢ_{ⱼ₋₁}-CPᵢ_{ⱼ} (in whole or in part), the dose is calculated using image CTⱼ. Note, however, that because there might be a need to treat the arc-field over multiple breath-hold cycles, some care may be observed as regards the indexes. Once j>n, one can do the mapping between the computed tomography images and the arc sectors by subtracting n (or its multiplication) from the index.

By one approach, these teachings can provide for proper evaluation of dose delivered for a patient undergoing deformations during the delivery by requiring that the dose delivered during different phases of the breath cycle should be deformed and summed into one selected phase. This can be done using a deformable registration function Ξ(CT₀, CTᵢ) that maps CTᵢ to a selected reference phase CT (here, the first phase CT₀ is selected, but it can be any 3D-CT from the same patient). When this deformation field is applied to a dose distribution, the result is the dose deformed to a reference phase coordinate system as follows:

$${\tilde{D}}_{l} = Ξ ( {CT}_{0} , {CT}_{i} ; {D}_{i} ) ,$$

where Dᵢ is dose calculated to CTᵢ and \tilde{D}_{ι} is its deformation to the selected reference image CT₀. There are multiple algorithms available for performing this deformable registration between two images. As these teachings are not overly sensitive to any particular choices in those regards, further details are not provided here for the sake of brevity.

Once the deformation field has been found, that information can be stored (for example, in the aforementioned memory 102). The deformation operation itself is relatively efficient and can even be differentiated in order to calculate gradient functions as desired.

With the aid of the deformation field, the total dose can be expressed as:

$${D}_{tot} = {\sum }_{i} Ξ ( {CT}_{0} , {CT}_{ci ( i )} ; D ( {F}_{i} , {CT}_{ci ( i )} ) ) ,$$

where Fᵢ is the fluence of fields delivered to the patient while the patient anatomy is assumed to be in the state described by i^{th} section. The summation can be done over the whole arc(s). Index ci(i) refers to which CT should be used for i^{th} arc-sector.

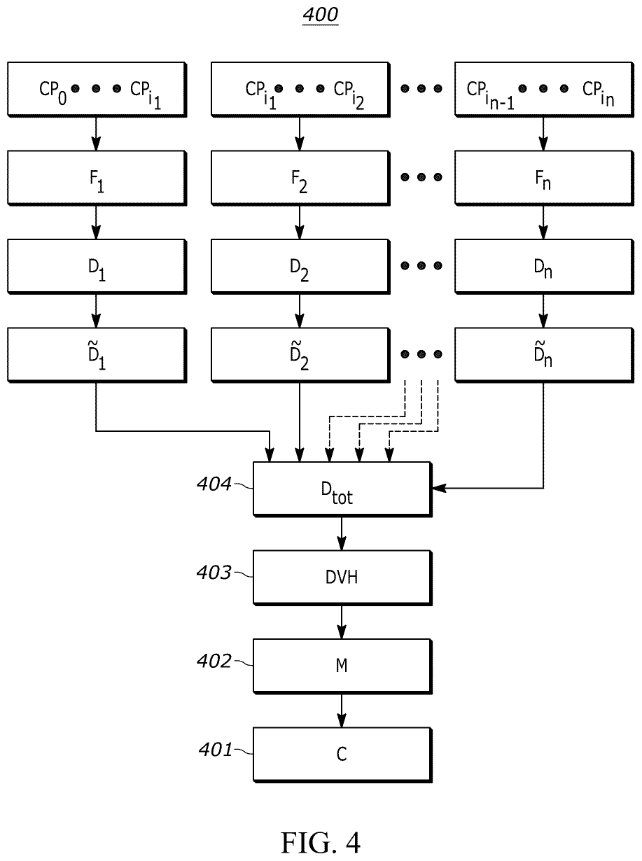

A total cost function can now be calculated using the update schema 400 presented in FIG. 4. Per this update schema 400, cost function C 401 through metric vectors M 402 can be calculated from the corresponding dose volume histogram(s) 403 which in turn were calculated based on the total dose Dₜₒₜ. The individual deformed doses can be calculated using separate deformation fields from different dose fields, where each different dose field is a function of the control points associated to a given corresponding phase.

These teachings can offer better treatment efficiency and/or accuracy. Compared to gating (alone) methods, these teachings allow treating the radiation treatment plan over a broader range of the breathing cycle. And compared to standard free-breathing planning (using a single leads image), the planning per these teachings is based on a more realistic estimation of the delivered dose. Breathing freely is also usually more comfortable for the patient.

Further aspects of the invention are provided by the subject matter of the following clauses.

Clause 1. A method to facilitate optimizing a radiation treatment plan for a particular patient, the method comprising: by a control circuit: accessing a plurality of images of the particular patient, which images are temporally dispersed and collectively depict a particular dynamically-moving part of the particular patient over time; optimizing a first part of the radiation treatment plan as a function of a first one of the plurality of images; optimizing a second, different part of the radiation treatment plan as a function of a second, different one of the plurality of images; outputting an optimized radiation treatment plan.

Clause 2. The method of clause 1 wherein the plurality of images are a plurality of computed tomography images.

Clause 3. The method of either of clause 1 and 2 wherein the particular dynamically-moving part of the particular patient comprises at least a part of particular patient's chest that moves as the particular patient breathes.

Clause 4. The method of any of clause 1 through 3 further comprising: separately optimizing each of N different parts of the radiation treatment plan, each as a function of a separate, different one of the plurality of images, where “N” is an integer greater than “2.”

Clause 5. The method of clause 4 wherein the first part of the radiation treatment plan corresponds to a first control point, the second part of the radiation treatment plan corresponds to a second, different control point, and each of the N different parts of the radiation treatment plan each correspond to different control points.

Clause 6. The method of any of clause 1 through 5 further comprising: processing the plurality of images to determine at least one dynamic feature of the particular dynamically-moving part of the particular patient over time to thereby select which of the plurality of images are to be used when optimizing different parts of the radiation treatment plan.

Clause 7. The method of any of clause 1 through 6 further comprising: administering the optimized radiation treatment plan to the particular patient.

Clause 8. The method of clause 7, wherein administering the optimized radiation treatment plan to the particular patient comprises synchronizing administration of the optimized radiation treatment plan with observed during-treatment movement of the particular dynamically-moving part of the particular patient.

Clause 9. The method of clause 8, wherein the movement of the particular dynamically-moving part of the particular patient comprises breathing-based movement of the particular patient's chest.

Clause 10. The method of clause 8 wherein the synchronizing administration of the optimized radiation treatment plan comprises selectively controlling gantry speed while administering the optimized radiation treatment plan.

Clause 11. An apparatus to facilitate optimizing a radiation treatment plan for a particular patient, the apparatus comprising: a control circuit configured to: access a plurality of images of the particular patient, which images are temporally dispersed and collectively depict a particular dynamically-moving part of the particular patient over time; optimize a first part of the radiation treatment plan as a function of a first one of the plurality of images; optimize a second, different part of the radiation treatment plan as a function of a second, different one of the plurality of images; output an optimized radiation treatment plan.

Clause 12. The apparatus of clause 11 wherein the plurality of images are a plurality of computed tomography images.

Clause 13. The apparatus of either of clause 11 or 12 wherein the particular dynamically-moving part of the particular patient comprises at least a part of particular patient's chest that moves as the particular patient breathes.

Clause 14. The apparatus of any of clause 11 through 13 wherein the control circuit is further configured to separately optimize each of N different parts of the radiation treatment plan, each as a function of a separate, different one of the plurality of images, where “N” is an integer greater than “2.”

Clause 15. The apparatus of clause 14 wherein the first part of the radiation treatment plan corresponds to a first control point, the second part of the radiation treatment plan corresponds to a second, different control point, and each of the N different parts of the radiation treatment plan each correspond to different control points.

Clause 16. The apparatus of any of clause 11 through 15 wherein the control circuit is further configured to: process the plurality of images to determine at least one dynamic feature of the particular dynamically-moving part of the particular patient over time to thereby select which of the plurality of images are to be used when optimizing different parts of the radiation treatment plan.

Clause 17. The apparatus of any of clause 11 through 16 wherein the control circuit is further configured to: administer the optimized radiation treatment plan to the particular patient.

Clause 18. The apparatus of clause 17, wherein the control circuit is configured to administer the optimized radiation treatment plan to the particular patient by synchronizing administration of the optimized radiation treatment plan with observed during-treatment movement of the particular dynamically-moving part of the particular patient.

Clause 19. The apparatus of clause 18, wherein the movement of the particular dynamically-moving part of the particular patient comprises breathing-based movement of the particular patient's chest.

Clause 20. The apparatus of clause 18 wherein the control circuit is configured to synchronize administration of the optimized radiation treatment plan by selectively controlling gantry speed while administering the optimized radiation treatment plan.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method to facilitate optimizing a radiation treatment plan for a particular patient, the method comprising:
by a control circuit:
accessing a plurality of images of the particular patient, which images are temporally dispersed and collectively depict a particular dynamically-moving part of the particular patient over time;
optimizing a first part of the radiation treatment plan as a function of a first one of the plurality of images;
optimizing a second, different part of the radiation treatment plan as a function of a second, different one of the plurality of images;
outputting an optimized radiation treatment plan.

2. The method of claim 1 wherein the plurality of images are a plurality of computed tomography images.

3. The method of claim 1 wherein the particular dynamically-moving part of the particular patient comprises at least a part of particular patient's chest that moves as the particular patient breathes.

4. The method of claim 1 further comprising:
separately optimizing each of N different parts of the radiation treatment plan, each as a function of a separate, different one of the plurality of images, where “N” is an integer greater than “2.”

5. The method of claim 4 wherein the first part of the radiation treatment plan corresponds to a first control point, the second part of the radiation treatment plan corresponds to a second, different control point, and each of the N different parts of the radiation treatment plan each correspond to different control points.

6. The method of claim 1 further comprising:
processing the plurality of images to determine at least one dynamic feature of the particular dynamically-moving part of the particular patient over time to thereby select which of the plurality of images are to be used when optimizing different parts of the radiation treatment plan.

7. The method of claim 1 further comprising:
administering the optimized radiation treatment plan to the particular patient.

8. The method of claim 7, wherein administering the optimized radiation treatment plan to the particular patient comprises synchronizing administration of the optimized radiation treatment plan with observed during-treatment movement of the particular dynamically-moving part of the particular patient.

9. The method of claim 8, wherein the movement of the particular dynamically-moving part of the particular patient comprises breathing-based movement of the particular patient's chest.

10. The method of claim 8 wherein the synchronizing administration of the optimized radiation treatment plan comprises selectively controlling gantry speed while administering the optimized radiation treatment plan.

11. An apparatus to facilitate optimizing a radiation treatment plan for a particular patient, the apparatus comprising:
a control circuit configured to:
access a plurality of images of the particular patient, which images are temporally dispersed and collectively depict a particular dynamically-moving part of the particular patient over time;
optimize a first part of the radiation treatment plan as a function of a first one of the plurality of images;
optimize a second, different part of the radiation treatment plan as a function of a second, different one of the plurality of images;
output an optimized radiation treatment plan.

12. The apparatus of claim 11 wherein the plurality of images are a plurality of computed tomography images.

13. The apparatus of claim 11 wherein the particular dynamically-moving part of the particular patient comprises at least a part of particular patient's chest that moves as the particular patient breathes.

14. The apparatus of claim 11 wherein the control circuit is further configured to separately optimize each of N different parts of the radiation treatment plan, each as a function of a separate, different one of the plurality of images, where “N” is an integer greater than “2.”

15. The apparatus of claim 14 wherein the first part of the radiation treatment plan corresponds to a first control point, the second part of the radiation treatment plan corresponds to a second, different control point, and each of the N different parts of the radiation treatment plan each correspond to different control points.

16. The apparatus of claim 11 wherein the control circuit is further configured to:
process the plurality of images to determine at least one dynamic feature of the particular dynamically-moving part of the particular patient over time to thereby select which of the plurality of images are to be used when optimizing different parts of the radiation treatment plan.

17. The apparatus of claim 11 wherein the control circuit is further configured to:
administer the optimized radiation treatment plan to the particular patient.

18. The apparatus of claim 17, wherein the control circuit is configured to administer the optimized radiation treatment plan to the particular patient by synchronizing administration of the optimized radiation treatment plan with observed during-treatment movement of the particular dynamically-moving part of the particular patient.

19. The apparatus of claim 18, wherein the movement of the particular dynamically-moving part of the particular patient comprises breathing-based movement of the particular patient's chest.

20. The apparatus of claim 18 wherein the control circuit is configured to synchronize administration of the optimized radiation treatment plan by selectively controlling gantry speed while administering the optimized radiation treatment plan.
